(19) United States
(12) Patent Application Publication
(10) Pub. No.: US 2009/0246247 A1
(43) Pub. Date: Oct. 1, 2009

(54) COMPOSITE ENTEROCYSTOPLASTY

(75) Inventors: Dhanuraj Shetty, Jersey City, NJ (US); Sridevi Dhanaraj, Raritan, NJ (US); Ziwei Wang, Monroe Twp., NJ (US); Jackie Jacobus Johannes Maria Donners, West Windsor, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Advanced Technologies and Regenerative Medicine, LLC, Raynham, MA (US)

(21) Appl. No.: 12/411,428

(22) Filed: Mar. 26, 2009

Related U.S. Application Data
(60) Provisional application No. 61/039,892, filed on Mar. 27, 2008.

(51) Int. Cl. A61F 2/00; A61K 35/38

(52) U.S. Cl. 424/423; 424/551

(57) ABSTRACT

The present invention relates to methods for tissue augmentation or regeneration. More specifically, the present invention provides for a composite enterocystoplasty procedure using a biocompatible scaffold and minced autologous tissue for implantation in a mammalian bladder.

### RELATED APPLICATIONS

This application is a non-provisional filing of a provisional application U.S. Pat. App. No. 61/039,892.

### FIELD OF THE INVENTION

The present invention relates to methods for tissue augmentation or regeneration. More specifically, the present invention provides for a composite enterocystoplasty procedure using a biocompatible scaffold and minced autologous tissue derived from bladder for implantation in a mammalian bladder.

### BACKGROUND

The current standard of care for augmenting or repairing congenital or acquired abnormalities of the bladder is the enterocystoplasty procedure, wherein a portion of intestine is cut, detubularized, and subsequently attached to the cystectomized bladder. Although this procedure has been a major advance in the treatment and outcomes for patients, the benefits are offset by well-documented, relatively common, and potentially serious complications. These include mucus production, stone formation, chronic low-grade infection, and metabolic disturbance, all of which are attributable to the lining of the intestine, which is an absorptive, mucus-secreting epithelium that is not adapted to prolonged contact with urine.

The use of seromuscular intestinal patches with the serosal side toward the bladder lumen showed encouraging results in rats, but their use in larger animals with either the serosa or the demucosalized side in contact with the urine in the bladder resulted in fibrosis and contraction of the patch. The ideal material for bladder reconstruction would combine the compliance afforded by the smooth muscle layer with the non-absorptive barrier lining of normal urothelium. Attempts to overcome these limitations by using other tissue sources have either met with limited success or have limited capacity due to the tissue source.

In order to avoid the complications of enterocystoplasty that are largely attributed to the epithelial layer of the intestine, other researchers have favored the concept of composite enterocystoplasty. In the composite enterocystoplasty procedure autologous urothelium is harvested from bladder tissue and cultured in vitro, and then later combined with de-epithelialized intestinal segments at the time of reconstruction. Augmentation of the enterocystoplasty procedure by the use of cultured cells has been described, such as by Fraser, et al. in BJU International 93:609-616 (2004), and by Oberpenning, et al. in Nature Biotechnology 17:149-155 (1998). However, in a clinical setting the major draw back of the cell culturing approach is that it is a two step process that requires the patient to undergo surgery for two separate procedures: one to harvest the biopsy for initiating the cell culture and isolation, and a second procedure for implantation of the graft. The use of cultured cells introduces additional steps that increase the time, cost, patient discomfort, and surgical risk of the procedure.

Thus, there remains a need for an effective treatment for the augmentation and repair of the bladder.

### SUMMARY OF THE INVENTION

An object of the present invention provides methods for the augmentation and repair of a mammalian bladder. Methods are disclosed comprising the use of a sample of autologous tissue from a healthy portion of the bladder to regenerate new bladder tissue using a modified composite enterocystoplasty procedure. As used herein, sample shall mean a biopsy or biopsied autologous tissue used in the invention. The healthy bladder tissue sample is minced and then used to populate a sample of de-epithelialized intestinal tissue that is subsequently used to augment or repair the bladder.

Another object of the present invention is to provide a method for the augmentation and repair of a mammalian bladder using a sample of de-epithelialized intestinal tissue having a polymer scaffold attached thereto, wherein the polymer scaffold is populated with the minced autologous tissue derived from the bladder, urethra, ureter, or buccal tissue.

It is another object of the present invention to provide a method for the augmentation and repair of a mammalian bladder using a sample of de-epithelialized intestinal tissue having a minced autologous tissue incorporated thereon, wherein the minced autologous tissue is comprised of urothelial tissue derived from the bladder, bladder, urethra, ureter, or buccal tissue.

It is another object of the present invention to provide a method for the augmentation and repair of a mammalian bladder using a sample of de-epithelialized intestinal tissue populated with minced bladder tissue, wherein the minced bladder tissue is held in place with an adhesive.

It is another object of the present invention to provide a method for the augmentation and repair of a mammalian bladder using a sample of de-epithelialized intestinal tissue populated with minced bladder tissue and an adhesive, wherein the minced bladder tissue is held in place using a polymer scaffold in the form of a mesh, knit, film, hydrogel, collagen, or a nonwoven.

### BRIEF DESCRIPTION OF THE DRAWINGS

FIG. 1a shows an image of a Hematoxylin & Eosin (H/E) stained section of native intestinal tissue section, with epithelium denoted by the arrow.

FIG. 1b shows an image of an H/E stained section of the intestinal tissue section following de-epithelialization.

FIG. 2 shows an image of an H/E stained section of de-epithelialized intestinal tissue following 3 days of in-vitro culture.

FIG. 3a shows an image of an H/E stained section of the assembled construct with minced bladder tissue and VICRYL mesh after 6 weeks subcutaneous implantation in a SCID mouse.

FIG. 3b shows an image of an H/E stained section of the assembled construct with minced urothelial tissue after 6 weeks subcutaneous implantation in a SCID mouse.

FIG. 4a shows an image of an H/E stained section of the assembled construct with minced bladder tissue after 6 weeks subcutaneous implantation in a SCID mouse.

FIG. 4b shows an image of an H/E stained section of the assembled construct with minced urothelium tissue after 6 weeks subcutaneous implantation in a SCID mouse.

FIG. 5 shows a section of one embodiment of the invention having a layer of minced urothelium tissue disposed between a layer of VICRYL mesh and the de-epithelialized intestine.

FIG. 6 shows a schematic of the composite enterocystoplasty procedure.

### DETAILED DESCRIPTION OF THE INVENTION

It should be understood that this invention is not limited to the particular methods, protocols, etc., described herein and, as such, may vary. The terminology used herein is for the purposes of describing particular embodiments only, and is not intended to limit the scope of the present invention, which is defined solely by the claims.

As used herein and in the claims, the singular forms “a”, “an”, and “the” include the plural reference unless the context clearly indicates otherwise. Thus, for example, a reference to a cell may be a reference to one or more such cells, including equivalents known to those skilled in the art unless the context of the reference clearly dictates otherwise. Unless defined otherwise, all technical terms used herein have the same meaning as those commonly understood to one of ordinary skill in the art to which this invention pertains. Other than in the operating examples, or unless otherwise indicated, all numbers expressing quantities of ingredients or reaction conditions used herein should be understood as modified by the term “about”.

As used herein, the term “minced tissue” refers to a sample of biological tissue that has been chopped, ground, sliced, cut, worked into a paste or otherwise reduced in minimum particle size from the native tissue state to having particles no larger than from about 50 microns to about 1 mm in size, and more preferably from about 200 microns to about 1 mm. The minced tissue contains tissue fragments, clumps or clusters of cells, individual whole cells, and may also contain a portion of ruptured cells. The cells liberated from the disrupted tissue by mincing are able to migrate through the surrounding environment.

As used herein, the term “bioresorbable polymer” refers to one that will break down into small segments when exposed to moist body tissue. The segments are then either absorbed or excreted by the body, either in their native state or as metabolized derivatives of their native state. More particularly, the biodegraded segments do not elicit a permanent chronic foreign body reaction because no permanent residue of the segment is retained by the body. The terms “biodegradable”, “bioresorbable”, “absorbable”, bioabsorbable”, and “resorbable” are equivalent and may be used interchangeably.

As used herein, the term “scaffold” refers to a sheet, disc, cylinder, tube, hollow sphere or spheroid, or portion thereof, or any shaped piece of biocompatible material or combination of biocompatible materials used to contain, carry, or deliver an amount of at least one minced tissue upon implantation into a mammal. The terms “matrix” and “carrier” are understood to be equivalent and synonymous with the term “scaffold”. In preferred embodiments, the shape of the scaffold would be a portion of a hollow sphere or spheroid. The scaffold can be made from biodegradable or non-biodegradable materials, or a combination of biodegradable and non-biodegradable materials, as well as foams, non-wovens, hydrogels or films. Furthermore, the scaffold can be configured and shaped to the desired size and shape before use, so as to conform to a defect site.

As used herein, the term “composite enterocystoplasty” refers to a surgical procedure wherein a sample of healthy intestinal tissue is obtained from a patient in need of bladder therapy, the epithelial layer is removed from the sample of healthy intestinal tissue and is replaced with autologous bladder cells or tissue before attaching the composite tissue device to the bladder of the patient, thereby augmenting or repairing the bladder. As used herein, the terms “denuded tissue” and “de-epithelialized tissue” are equivalent and understood to refer to a sample of intestinal tissue wherein the epithelial layer is removed from the tissue, leaving only substantially the smooth muscle layer. Furthermore, when referring to denuded or de-epithelialized tissue, it is understood that any discussion thereof refers to the side of the intestinal tissue that has had the epithelial layer removed, and not to the native smooth muscular side of the tissue, unless expressly stated otherwise.

All patents and other publications identified are incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention provides for methods of repairing or augmenting a mammalian bladder comprising a composite enterocystoplasty procedure using minced bladder tissue. The method may further include the use of an adhesive material to retain the minced bladder tissue in place, and may further include the use of a polymer scaffold attached to the intestinal tissue.

The problems associated with the current methods of the enterocystoplasty procedure are due to the epithelial layer of the intestinal segment, which is an absorptive, mucus-secreting epithelium. Simply removing this epithelial layer results in fibrosis and contraction of the implanted tissue. Removing this epithelial layer and replacing it with cultured urothelium cells is known in the art, but requires additional surgery and time for the in vitro culturing of the cells. By using a minced bladder tissue to populate the de-epithelialized intestinal segment, we have overcome the limitations of the prior art. The minced bladder tissue used to populate the de-epithelialized intestinal segment can be a combination of smooth muscle tissue and urothelial tissue, or it can be urothelial tissue alone. The minced bladder tissue serves as a source of cells to adhere to, migrate, proliferate, and populate the de-epithelialized side of the intestinal patch, thereby creating a urothelial layer that will keep the urine contained within the bladder without the complications seen with an intestinal epithelial layer.

The source of bladder tissue can be obtained during the same surgery when the bladder is being treated. Thus, in one embodiment of the present invention the bladder of a patient in need of bladder augmentation has an incision made in the bladder and a small portion of the bladder tissue is removed from the incision area. The isolated portion of bladder tissue is then minced into a fine paste, for example by repeated slicing with a scalpel, applied to a de-epithelialized segment of intestine, which is then attached to the bladder to increase the size of the bladder. This process can all be performed within the scope of a single surgery, thereby reducing the time, risk, and discomfort of additional surgery, and furthermore ensuring that only autologous tissue is implanted in the recipient.

In another embodiment of the present invention, the bladder tissue sample obtained from the patient can further be prepared by using a scalpel to remove the urothelial layer from the smooth muscle layer, such as by scraping. By mincing only the urothelial layer for subsequent application to the de-epithelialized segment of intestine, the population of cells used to create the urothelial layer is more homogenous and uniform of the desired cell type, and may provide for a faster generation of a continuous urothelial layer.

In another embodiment of the present invention a biocompatible adhesive material is used to hold the minced tissue in place. Suitable adhesive materials include hydrogels including high molecular weight hyaluronic acid, collagen gel, and fibrin glue. These materials have good biocompatibility and provide a cell-friendly environment, as well as having a high viscosity to provide adhesion between the minced tissue and the de-epithelialized intestinal tissue. Thus, after spreading the minced tissue onto the de-epithelialized intestinal segment, an adhesive material is spread over the minced tissue to facilitate maintaining it in place. Alternatively, the adhesive material could be mixed with the minced tissue before application to the de-epithelialized intestine segment, and then applied as a mixture.

In another embodiment of the present invention a biocompatible scaffold is attached to the de-epithelialized intestine segment. The scaffold could be attached to the intestine segment prior to the application of the minced tissue, or the scaffold could be attached after the application of the minced tissue to the scaffold. The means of attachment of the scaffold could be sutures, staples, or adhesives, or a combination thereof. Suitable polymer scaffolds could be biodegradable foams, non-wovens, mesh, knits, hydrogels or films.

One skilled in the art will appreciate that the selection of a suitable material for forming the biocompatible scaffold used in the present invention depends on several factors. These factors include in vivo mechanical performance; cell response to the material in terms of cell attachment, proliferation, migration and differentiation; biocompatibility; and optionally, bioabsorption (or bio-degradation) kinetics. Other relevant factors include the chemical composition, spatial distribution of the constituents, the molecular weight of the polymer, and the degree of crystallinity. A variety of biocompatible polymers can be used to make the scaffold according to the present invention, including synthetic polymers, natural polymers or combinations thereof.

The term “natural polymer” refers to polymers that are naturally occurring. Suitable biocompatible natural polymers include those known in the art, and include, but are not limited to, collagen, elastin, thrombin, silk, keratin, fibronectin, starches, poly(amino acid), gelatin, alginate, pectin, fibrin, oxidized cellulose, chitin, chitosan, tropoelastin, hyaluronic acid, ribonucleic acids, deoxyribonucleic acids, polypeptides, proteins, polysaccharides, polynucleotides and combinations thereof.

As used herein the term “synthetic polymer” refers to polymers that are not found in nature, even if the polymers are made from naturally occurring biomaterials. Suitable biocompatible synthetic polymers can include, but are not limited to, hydrogels, aliphatic polyesters, poly(amino acids), copoly(ether-esters), polyalkylene oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, polyurethanes, polycaprolactones, polydioxanones, poly(ether urethanes), poly(ester urethanes), poly(propylene fumarate), poly(hydroxyalkanoate), and blends or copolymers thereof. Exemplary synthetic biocompatible polymers include polylactic acid (PLA) and polyglycolic acid (PGA), and combinations thereof, such as are commonly known in the art. Suitable synthetic polymers for use in the present invention can also include biosynthetic polymers based on sequences found in collagen, elastin, thrombin, silk, keratin, fibronectin, starches, poly(amino acid), gelatin, alginate, pectin, fibrin, oxidized cellulose, chitin, chitosan, tropoelastin, hyaluronic acid, ribonucleic acids, deoxyribonucleic acids, polypeptides, proteins, polysaccharides, polynucleotides and combinations thereof.

FIG. 5 shows a section of a composite patch of material to be used in the present invention, comprised of a section of de-epithelialized intestine having a layer of minced urothelial tissue disposed thereon, and further having a polymer scaffold maintaining the minced urothelial tissue in proximity to the de-epithelialized intestine section.

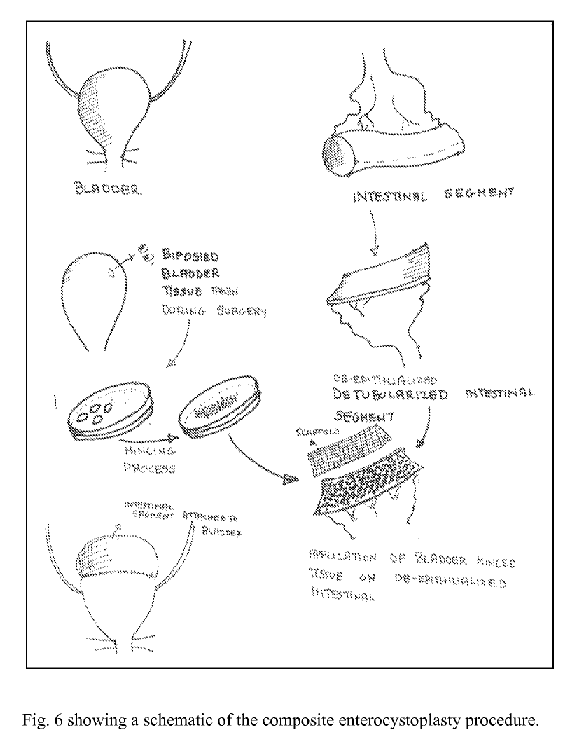

FIG. 6 shows a schematic of the basic composite enterocystoplasty procedure, wherein a portion of healthy bladder tissue is harvested and minced into a fine paste to provide a source of viable cells that are spread on a section of de-epithelialized intestine, which is then attached to a bladder to repair the bladder. Optionally an adhesive and a polymer scaffold can be used.

The following examples are meant only to be illustrative in nature of the present invention, and not to be limiting in scope. One skilled in the art would easily conceive of other embodiments that would be considered within the scope of the present invention.

### Example 1

In this example we investigated the ability of our composite enterocystoplasty method utilizing porcine tissues in a SCID mouse model. We utilized four different sample preparations for comparison: a) minced whole bladder tissue applied to a de-epithelialized intestinal tissue and secured with fibrin glue, b) minced bladder urothelial tissue applied to a de-epithelialized intestinal tissue and secured with fibrin glue, c) minced whole bladder tissue applied to a de-epithelialized intestinal tissue as in a) and further held in place with VICRYL mesh, d) minced bladder urothelial tissue applied to a de-epithelialized intestinal tissue as in b) and further held in place with VICRYL mesh. Sections of de-epithelialized intestinal tissue alone were implanted into SCID mice as controls and evaluated for re-growth of the intestinal epithelial layer.

Healthy intact bladder tissue and healthy intestinal tissue were obtained from a porcine source. The bladder tissue was dissected open and the intravesicular fluid within the bladder was aspirated out. The intestinal tissue was also dissected open. Sample pieces of bladder and intestinal tissues approximately 6×4 cm were washed separately in phosphate buffered saline (PBS) containing antibiotic antimycotic solution, and used for the experiment. A six (6) mm diameter full thickness biopsy was obtained from the bladder tissue and minced finely to a paste. Similarly, a 6 mm diameter full thickness biopsy was obtained from the bladder tissue and the urothelial layer was scraped off using a scalpel blade; this urothelial layer was then minced and used for the experiment.

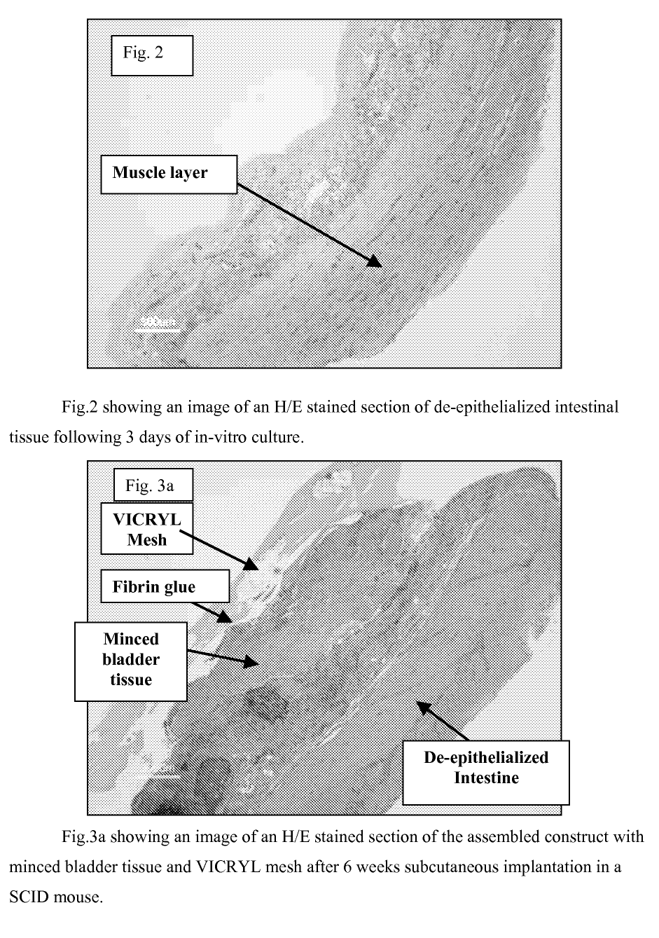

The intestinal tissue was de-epithelialized using a surgical blade by gentle scrapping along the length of the inner surface of the intestine. Tissue was rinsed in PBS prior to use. Several 6 mm diameter biopsy punches were made from the de-epithelialized intestinal segment and used for the experiment. The biopsy punches of de-epithelialized intestinal tissue were cultured in standard culture medium at 37° C. for 3 days. H/E stained sections showed that the intestinal segments remained viable with no outgrowth of the intestinal epithelium cells after 3 days of culture (see FIG. 2).

Constructs were assembled by distributing 14 mg of minced bladder tissue or minced urothelial tissue, over the 6 mm diameter biopsy samples of de-epithelialized intestinal tissue. The minced tissue was held in place with the help of 6 microliters of fibrin glue. In some samples the minced tissue was further stabilized by placing a 6 mm punch of absorbable VICRYL mesh over the minced tissue. The composite constructs were then implanted subcutaneously into SCID mice for 6 weeks.

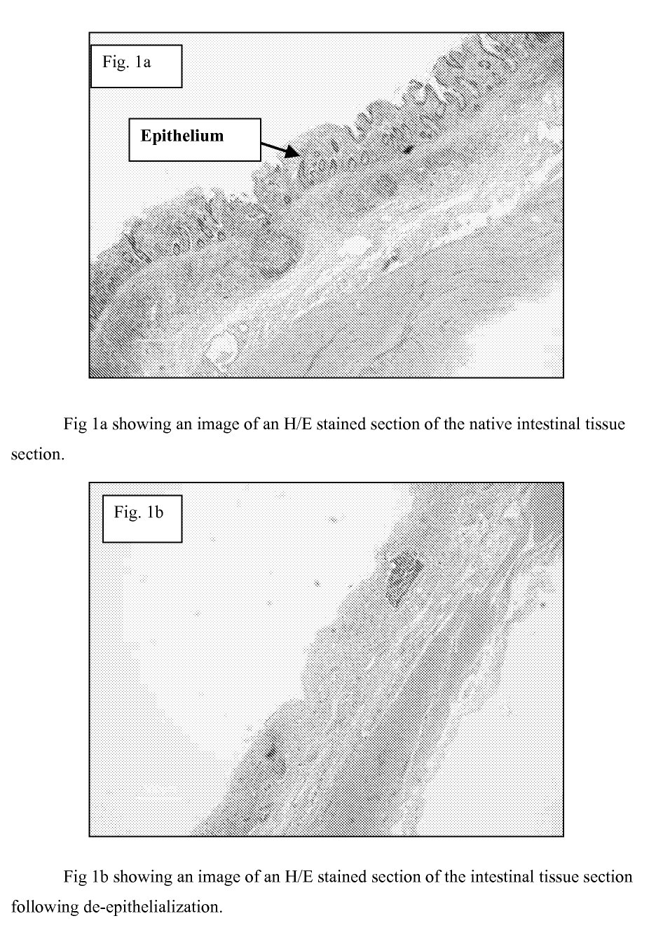

FIG. 1a shows an image of an H/E stained section of normal or native porcine intestine with the normal epithelium intact. FIG. 1b shows an image of an H/E stained section of the de-epithelialized porcine intestine after the removal of the epithelium. FIG. 2 shows an image of an H/E stained section of the de-epithelialized intestinal tissue after 3 days of culturing in-vitro in standard growth serum. The image shows that there is no re-growth of the normal intestinal epithelium.

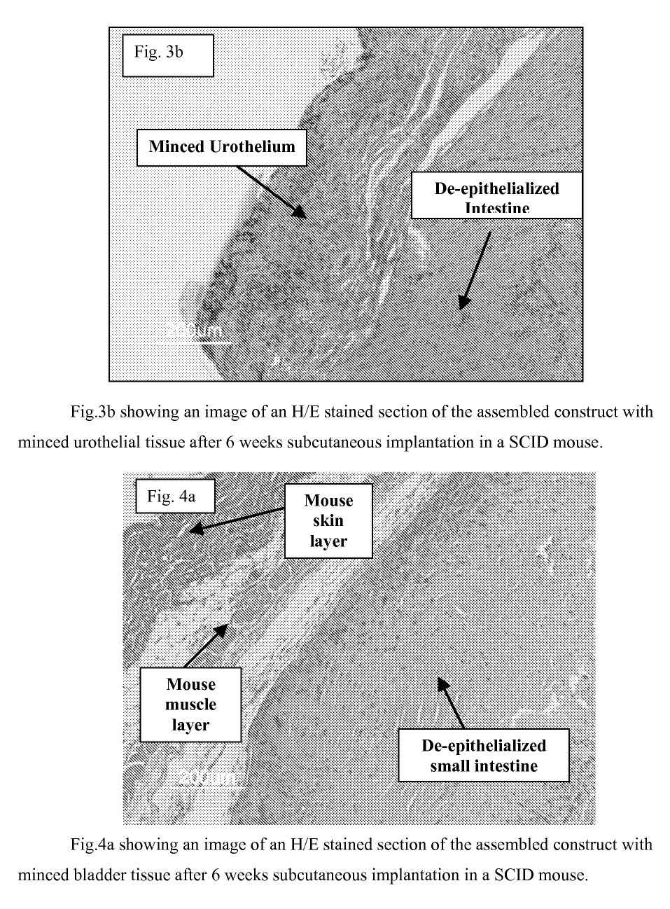

FIG. 3a shows an image of an H/E stained section of the construct using whole minced bladder held in place on the de-epithelialized intestine using VICRYL mesh and fibrin glue after 6 weeks of subcutaneous implantation in a SCID mouse.

FIG. 3b shows an image of an H/E stained section of the construct using minced urothelial held in place on the de-epithelialized intestine using fibrin glue after 6 weeks of subcutaneous implantation in a SCID mouse.

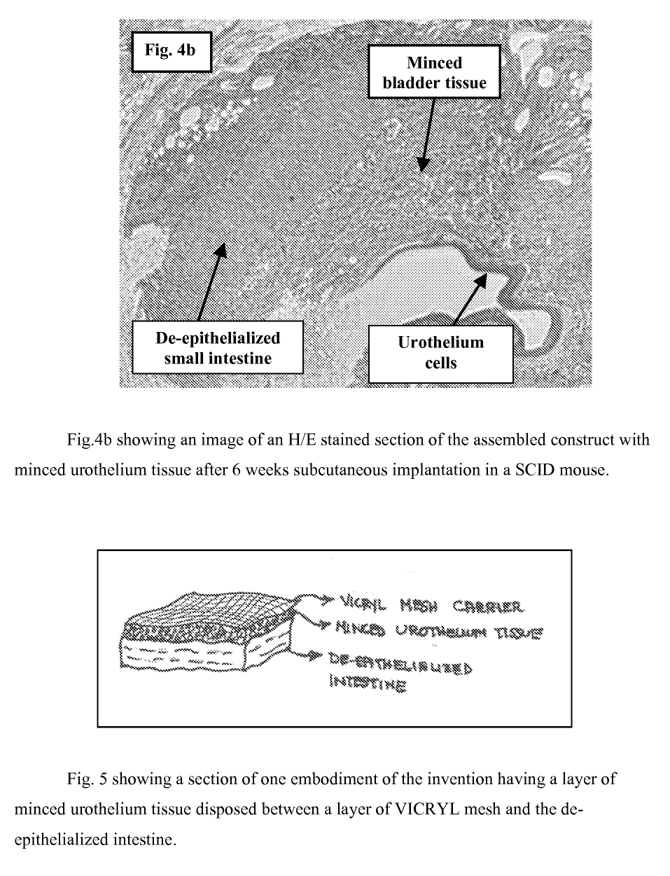

FIG. 4a shows an image of an H/E stained section of a de-epithelialized intestine control sample using no minced tissue after 6 weeks of subcutaneous implantation in a SCID mouse. The image shows no re-growth of the intestinal epithelium.

FIG. 4b shows an image of an H/E stained section of the construct using minced urothelial held in place on the de-epithelialized intestine using fibrin glue after 6 weeks of subcutaneous implantation in a SCID mouse. The image shows a layer of urothelial tissue (cells) that formed above the de-epithelialized intestinal tissue.

The histology analysis of these sections demonstrate the viability and robustness of the composite enterocystoplasty procedure of the present invention, showing robust growth and attachment of the desired cell populations, without any signs of undesired re-growth of the intestinal epithelium.

### Example 2

A patient in need of bladder augmentation therapy is prepared for surgery as is commonly known in the art. A 15 cm segment of the intestine is removed from the patient and the continuity of the intestine is re-established by an end-to-end two-layer anastomosis with sutures. The isolated intestinal segment is cut open and the epithelial layer of the segment is removed by scraping with a scalpel. The de-epithelialized intestinal tissue segment is washed in PBS and then shaped and cut to the desired size to treat the bladder. A hollow spheroid shape would be created if desired by cutting and removing a portion of the intestinal tissue segment and suturing the edges together.

A portion of healthy autologous bladder tissue is removed from the patient and is minced using a scalpel or an appropriate mincing device to produce a fine paste comprised of smooth muscle cells, urothelial cells, and bladder tissue fragments having sizes ranging from about 50 microns to about 1 millimeter. The minced tissue paste is then spread evenly over the de-epithelialized surface of the intestinal segment. The composite intestinal segment is then further cut to the desired size and shape as needed and sutured into place on the bladder with the minced tissue side facing the lumen of the bladder, thereby providing an augmented bladder.

### Example 3

As in example 2, a patient is prepared for surgery and a segment of intestine is removed, de-epithelialized, and cut to the desired shape and size. A portion of healthy bladder tissue is also removed as in example 2. The urothelial tissue layer is removed from the isolated bladder tissue by scraping with a scalpel, and the urothelial tissue is minced into a fine paste using a scalpel or an appropriate mincing device. The minced urothelial tissue is applied to the de-epithelialized intestinal tissue, which is then implanted into the patient as in example 2, thereby providing an augmented bladder.

### Example 4

As in example 2, a patient is prepared for surgery and a segment of intestine is removed, de-epithelialized, and cut to the desired shape and size. A portion of healthy bladder tissue is also removed as in example 2. A sample of minced bladder tissue is further prepared as in example 2. The minced tissue paste is then spread evenly over the de-epithelialized surface of the intestinal segment, followed by a coating of fibrin glue. The composite tissue implant is then sutured into place on the bladder as in example 2, thereby providing an augmented bladder.

### Example 5

A patient is prepared for surgery and a segment of intestine is removed, de-epithelialized, and shaped to size as in example 2, and a sample of minced bladder tissue is further prepared as in example 2. A polymer scaffold comprised of 90/10 PGA/PLA is attached to the de-epithelialized intestinal segment using sutures. The minced tissue paste is then spread evenly over the polymer scaffold, followed by a coating of hydrogel. The composite tissue implant is then sutured into place on the bladder, as in example 2, thereby providing an augmented bladder.

### Example 6

A patient is prepared for surgery and a segment of intestine is removed, de-epithelialized, and shaped to size as in example 2, and a sample of minced bladder tissue is further prepared as in example 2. The minced tissue paste is then spread evenly over both surfaces of a polymer scaffold comprised of 90/10 PGA/PLA, followed by a coating of fibrin glue. The polymer scaffold is then attached to the de-epithelialized intestinal segment using sutures, and the composite tissue implant is then sutured into place on the bladder, as in example 2, thereby providing an augmented bladder.

Although this invention has been described with reference to specific embodiments, variations and modifications of the methods and means for repairing or augmenting a mammalian bladder will be readily apparent to those skilled in the art. Such variations and modifications are intended to fall within the scope of the appended claims.

We claim:

1. A method of repairing a mammalian bladder having an inner urothelial surface and an outer smooth muscular surface comprising the steps of,
a. obtaining a sample of healthy intestinal tissue,
b. removing the epithelial layer from said intestinal tissue sample,
c. obtaining a sample of healthy autologous bladder tissue,
d. mincing said bladder tissue sample,
e. applying said minced bladder tissue to the de-epithelialized side of said intestinal tissue, and
f. attaching said de-epithelialized intestinal tissue containing said minced tissue sample to said bladder such that the minced bladder tissue surface is in contact with and continuous with the inner urothelial surface of said bladder.

2. The method of claim 1 wherein said minced bladder tissue has a particle size of from about 50 microns to about 1 millimeter.

3. The method of claim 1 further comprising the steps of:
a. isolating urothelial tissue from said bladder tissue sample,
b. mincing said urothelial tissue,
c. applying said minced urothelial tissue sample to the de-epithelialized side of said intestinal tissue, and
d. attaching said intestinal tissue with said minced urothelial tissue sample to said bladder.

4. The method of claim 3 wherein the urothelial tissue sample is obtained by scraping the bladder tissue sample with a scalpel.

5. The method of claim 1 further comprising the step of applying an adhesive to said applied minced tissue.

6. The method of claim 5 wherein said adhesive is selected from the group consisting of hydrogel, hyaluronic acid, collagen gel, and fibrin glue.

7. The method of claim 6 wherein said adhesive is fibrin glue.

8. The method of claim 1 further comprising the step of attaching a polymer scaffold to said de-epithelialized intestinal tissue sample.

9. The method of claim 8 wherein said polymer scaffold is selected from the group consisting of a mesh, knit, film, hydrogel, collagen, and a nonwoven.

10. The method of claim 9 wherein the polymer scaffold is a mesh.
